(19) United States
(12) Patent Application Publication
(10) Pub. No.: US 2012/0021960 A1
(43) Pub. Date: Jan. 26, 2012

(54) AQUEOUS HAIR AND SKIN CLEANSING COMPOSITIONS

(75) Inventors: Hans Henning Wenk, Muelheim an der Ruhr (DE); Sascha Herrwerth, Essen (DE); Klaus Jenni, Essen (DE); Christian Noethe, Essen (DE)

(73) Assignee: EVONIK GOLDSCHMIDT GMBH, Essen (DE)

(21) Appl. No.: 13/260,657

(22) PCT Filed: Mar. 1, 2010
(86) PCT No.: PCT/EP2010/052519

§ 371 Date: Sep. 27, 2011

(30) Foreign Application Priority Data
Mar. 27, 2009 (EP) 09156406.2

(51) Int. Cl. A61K 8/46; A61K 8/898; A61Q 19/10; A61K 8/89; A61Q 5/02

(52) U.S. Cl. 510/122; 510/130; 510/127; 510/125

(57) ABSTRACT

The present invention relates to an aqueous composition comprising: i) 0.5 to 10 mass percent of at least one sulfo-succinate surfactant; ii) 0.5 to 10 mass percent of at least one amphoteric surfactant; and iii) free, OH-functional compounds similar to the alcohol component of the esters of the sulfosuccinate surfectants that are employed, wherein the mass ratio of sulfosuccinate surfectant i) to the free, OH-functional compound ranges from 100:1 to 100:20. The invention further relates to the use of such a composition for producing cleansing compositions, or as cleansing compositions, in particular for the cleansing of skin and hair, and to skin and/or hair cleansing agents containing such a composition.

The present invention relates to an aqueous composition which comprises at least one sulfosuccinate surfactant, at least one amphoteric surfactant, and at least one free, OH-functional compound, where these correspond to the alcohol component of the esters of the sulfosuccinate surfactants used.

For a long time there has been a need to provide mild cleansing compositions which are especially mild to skin and hair. Such cleansing compositions should not only be mild to skin and hair, but also have further desirable properties, such as e.g. good storage stability and good foaming behavior. Also desirable are care properties with regard to skin and hair.

Numerous mild surfactant systems have already been presented with which it has been attempted to produce corresponding cleansing compositions. The mild surfactant systems often exhibited a lower foaming ability, meaning that a larger addition of surfactant system became necessary, which in turn led to lower mildness of the product.

Suitable surfactant systems which lead to mild cleansing compositions with a useful foaming behavior which have been presented are those which comprise sulfosuccinates as surfactants in combination with amphoteric surfactants. Such systems are described inter alia in EP 1771148 and EP 1771538.

A disadvantage of sulfosuccinate systems was their unpredictable behavior with regard to viscosity adjustment and storage stability, in particular their tendency toward gel formation. According to EP 1771538, the hydrolysis products of the sulfosuccinates are responsible for the poor storage stability of such cleansing compositions.

EP 1771538 therefore proposes using sulfosuccinate systems which, besides the sulfosuccinate surfactant and an amphoteric surfactant, have at least 4% by weight of sulfosuccinic acid or a salt thereof. By adding sulfosuccinic acid or a salt thereof, it was possible to prevent an increase in the viscosity during storage. However, it was at the same time recognized that sulfosuccinic acid or a salt thereof is not suitable for adjusting the viscosity, but only causes a viscosity once set not to change or to change only slowly upon prolonged storage. Moreover, the presence of free acid leads to an increase in the electrolyte content, which has a disadvantageous effect on the surfactant system, e.g. the solubility of nonpolar substances is reduced.

Besides the problems with the storage stability of sulfosuccinate systems, however, there is also the problem of adjusting the viscosity of this system. In this regard, EP 1771538 details how sulfosuccinic acid or salts thereof have only a marginal effect on the adjustment of the viscosity and can consequently not be used as traditional viscosity regulators (thickeners).

It was therefore an object of the present invention to provide a sulfosuccinate surfactant system which can be adjusted to desired viscosities by simple means.

Surprisingly, it has been found that the viscosity of a sulfosuccinate surfactant system can be easily adjusted through the addition of from 1 to 20% by mass, based on the mass of sulfosuccinate surfactant, of free, OH-functional compound, where these correspond to the alcohol component of the esters of the sulfosuccinate surfactants used, in order to obtain the sulfosuccinate surfactant.

The present invention therefore provides aqueous compositions comprising:
i) from 0.5 to 10% by mass, preferably from 1 to 7.5% by mass, preferably from 2.5 to 5% by mass, of at least one sulfosuccinate surfactant,
ii) from 0.5 to 10% by mass, preferably from 1 to 7.5% by mass, preferably from 2.5 to 5% by mass, of at least one amphoteric surfactant, and
iii) OH-functional compounds, where these are like the alcohol component of the esters of the sulfosuccinate surfactants used,
   where the mass ratio of sulfosuccinate surfactant i) to free, OH-functional compound iii) is from 100:1 to 100:20, preferably from 100:3 to 100:15 and preferably from 100:5 to 100:12, and also the use thereof for producing cleansing compositions or as cleansing compositions, especially for the cleansing of skin and hair.

Moreover, the present invention provides skin cleansing compositions and/or hair cleansing compositions which have the corresponding compositions according to the invention.

The compositions according to the invention have the advantage that the viscosity of the composition can be easily adjusted. In particular, by adding the specified OH-functional compound it is possible to improve the effect of conventional thickeners and thus to reduce the required fraction of conventional thickeners.

A further advantage of the composition according to the invention is the improved foaming behavior, which is achieved through the presence of free, special OH-functional compounds.

If, besides the free OH compound, free sulfosuccinic acid is also present in the composition, as well as the simple adjustment of the viscosity, it is also possible to achieve better long-term stability of the composition or cleansing compositions comprising these.

The present invention is described by way of example below, without intending to limit the invention to these exemplary embodiments. Where ranges, generally formulae or compound classes are stated below, then these are intended to include not only the corresponding ranges or groups of compounds that are explicitly mentioned, but also all part ranges and part groups of compounds which can be obtained by removing individual values (ranges) or compounds. Where documents are cited in the course of the present description, then their content, in its entirety, is deemed to form part of the disclosure of the present invention. Unless stated otherwise, all percentage data below are data in % by mass and all average data is number-average data.

Within the context of the present invention, wherever it is stated that the free OH-functional compounds are like the alcohol components of the esters of the sulfosuccinate surfactants used, this should be understood as meaning that the free (not esterified with sulfosuccinic acid) OH-functional compounds have a composition which corresponds substantially to the composition of the alcohol component of the esters of the sulfosuccinates used. If the alcohol component of the sulfosuccinates is e.g. a fatty alcohol ethoxylate with a certain molar mass/alkoxylation distribution, then the free OH-functional compounds which are additionally present in the composition are likewise fatty alcohol ethoxylates with substantially identical molar mass/alkoxylation distribution. The amphoteric surfactants of component ii) are not sulfosuccinate surfactants iii). The free (not bonded to sulfosuccinic acid), OH-functional compounds are not amphoteric surfactants ii) or sulfosuccinate surfactants i).

According to the present invention, the aqueous composition is characterized in that it comprises
i) from 0.5 to 10% by mass, preferably from 1 to 7.5% by mass, preferably from 2.5 to 5% by mass, of at least one sulfosuccinate surfactant (sulfosuccinic acid ester surfactant),
ii) from 0.5 to 10% by mass, preferably from 1 to 7.5% by mass, preferably from 2.5 to 5% by mass, of at least one amphoteric surfactant, and
iii) free (not bonded to sulfosuccinic acid), OH-functional compounds, where these are like the alcohol component of the esters of the sulfosuccinate surfactants used,
   where the mass ratio of sulfosuccinate surfactant i) to free OH-functional compound iii) is from 100:1 to 100:20, preferably from 100:3 to 100:15 and preferably from 100:5 to 100:12.

The mass ratio of sulfosuccinate surfactant to amphoteric surfactant is preferably 4:1 to 1:4, preferably 3:1 to 1:3, and particularly preferably 2:1 to 1:2.

The sulfosuccinate surfactant is preferably a compound of the formula (Ia) or (Ib) or a mixture thereof. Customary sulfosuccinates usually consist of a mixture of the two isomers, where the mass fraction of compounds of the formula (Ib) predominates.

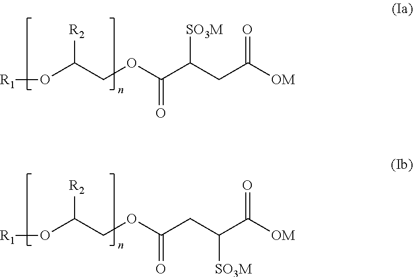

where
R¹=hydrocarbon radical, preferably hydrocarbon radical having on average 1 to 25, preferably 8 to 22, preferably 10 to 14, carbon atoms. Particularly preferably, R¹ is a branched or unbranched, preferably unbranched alkyl radical, preferably having the specified number of carbon atoms.
R²=H or C₁ to C₄ alkyl, preferably H or methyl, preferably H,
n=0 to 10, preferably 1 to 6, preferably 2 to 5 and particularly preferably about 3, where n is an average value (weight-average?),
M=identical or different cations, preferably alkali metal ions, preferably potassium or sodium ions or ammonium ions or alkanolammonium ions, preferably mono-, di- or triethanolammonium ions.

Preferred sulfosuccinate surfactants are mixtures of the compounds of the formula (Ia) or (Ib) in which compounds where n=0 are present in the mixture to 10 to 30% by weight, preferably to 15 to 25% by weight, where n=1 to 8 to 20% by weight, preferably to 11 to 16% by weight, where n=2 to 8 to 20% by weight, preferably to 12 to 16% by weight, where n=3 to 8 to 20% by weight, preferably to 13 to 15% by weight, where n=4 to 7 to 18% by weight, preferably to 10 to 13% by weight, where n=5 to 5 to 15% by weight, preferably to 7 to 10% by weight, where n=6 to 2 to 12% by weight, preferably to 5 to 8% by weight, where n=7 to 1 to 10% by weight, preferably to 3 to 6% by weight, where n=8 or higher to 0 to 15% by weight, preferably to 5 to 10% by weight. The distribution of the building blocks marked by the index n can be ascertained by means of gas chromatography following hydrolysis (the values for n specified in this section are not average values, but exact values).

Particularly preferred sulfosuccinate surfactant mixtures are those in which less than 2 mol % of compounds of the formula (Ia) or (Ib) where R¹=alkyl radical having 10 or fewer carbon atoms, 65-75 mol % of compounds of the formula (Ia) or (Ib) where R¹=alkyl radical having 12 carbon atoms, 20-30 mol % of compounds of the formula (Ia) or (Ib) where R¹=alkyl radical having 14 carbon atoms and less than 2 mol % of compounds of the formula (Ia) or (Ib) where R¹=alkyl radical having 16 and more carbon atoms are present.

The OH-functional compounds are preferably selected from the group comprising alcohols having 1 to 25, preferably 8 to 22, preferably 10 to 14, carbon atoms, or polyether alcohols of these alcohols. Preferred OH-functional compounds are mixtures comprising less than 2 mol % of alcohols having 10 or fewer carbon atoms in the alkyl chain, 65 to 75 mol % of alcohols having 12 carbon atoms in the alkyl chain, 20 to 30 mol % of alcohols having 14 carbon atoms in the alkyl chain and fewer than 2 mol % of alcohols having 16 and more carbon atoms in the alkyl chain. Preferably, the OH-functional compounds have polyether groups having on average up to 10, preferably 0 to 10, preferably 1 to 6, particularly preferably 2.5 to 3.5 or 4.5 to 5.5, very particularly preferably about 3 alkoxy units, preferably ethylene oxide units, where n is an average value (weight-average). Preferred OH-functional compounds are in the form of mixtures in which the composition with regard to the alcohols having a different number of building blocks with the index n corresponds to that for the preferred sulfosuccinate surfactant mixtures.

Preferred OH-functional compounds are mixtures of ethoxylated lauryl and myristyl alcohols which preferably have on average 1 to 6 ethylene oxide units in the polyether group. Particularly preferred OH-functional compounds are selected from the group comprising lauryl alcohol, PEG-5 lauryl alcohol, a mixture of PEG-3 alkyl alcohols, PEG-3 lauryl alcohol or a mixture of PEG-3 alkyl alcohols (in each case based on the average value of the polyether distribution), where the mixture preferably has less than 2 mmol % of alkyl alcohols having 10 or fewer carbon atoms in the alkyl chain, from 65 to 75 mol % of alkyl alcohols having 12 carbon atoms in the alkyl chain, from 20 to 30 mol % of alkyl alcohols having 14 carbon atoms in the alkyl chain and less than 2 mol % of alkyl alcohols having 16 or more carbon atoms in the alkyl chain.

Preferably, the mixture of PEG 3 alkyl alcohols has exclusively PEG-3 alkyl alcohols which are based on alkyl alcohols which have an even number of carbon atoms.

The OH-functional compounds can enter the composition according to the invention in different ways. Preferably, the OH functional compound enters as a result of addition of the same to the composition according to the invention.

It may be advantageous if the pH of the composition according to the invention has a value of from 4 to 7, preferably 4.5 to 6 and particularly preferably 4.5 to 5.75. To adjust the pH, it may be advantageous to add a suitable acid buffer to the composition according to the invention. Preference is given to using an acid buffer which has an acid buffer capacity of at least 0.01 mol of acid or H⁺ ion, preferably at least 0.02 mol of acid and particularly preferably 0.03 mol of acid per 1 of composition. Suitable buffer systems are e.g. those based on citric acid or polyacrylic acid, neutralized with sodium hydroxide or ammonium hydroxide. Preferably, the according to the invention has a pH of from 4 to 7, preferably 4.5 to 6 and particularly preferably >4.5 to 5.75, and moreover preferably an acid buffer capacity of 0.02 mol of acid per liter of composition.

Amphoteric surfactants which may be present in the composition according to the invention are in principle all amphoteric surfactants.

Preferably, the composition according to the invention has an amphoteric surfactant selected from the group comprising betaines, amphoacetates, amphodiacetates, hydroxysuitaines, amine oxides, amphopropionates and mixtures thereof.

Preferred betaines are those of the formulae (IIa) or (IIb)

R³—N⁺(CH₃)₂—CH₂—COO⁻ (IIa)

R⁵—C(O)—NH—CH₂—CH₂—CH₂—N⁺(CH₃)₂—CH₂—COO⁻ (IIb)

where R⁵=branched or unbranched, saturated or unsaturated, preferably unbranched alkyl radicals having 7 to 17 carbon atoms and R³ is an alkyl or alkylamidoalkyl radical, where the alkyl radical here may be branched or unbranched, preferably unbranched, and preferably has from 8 to 18, carbon atoms.

As betaine, the composition according to the invention preferably has a C₈₋₁₈ alkylbetaine, or a C₈₋₁₈ alkylamido-propylbetaine or a mixture thereof. Particularly preferably, betaines selected from oleylbetaine, caprylamidopropylbetaine, capramidopropylbetaine, laurylbetaine, laurylamidopropyl-betaine, isostearylamidopropylbetaine, cocobetaine, cocamido-propylbetaine, ricinoleamidopropylbetaine are present in the compositions according to the invention.

A further group of suitable amphoteric surfactants are the hydroxysultaines (formula (III)). In accordance with CTFA, this term is understood as meaning sulfobetaines which contain a hydroxypropylsulfonate group. Hydroxysultaines can be obtained e.g. by reacting tertiary amines with epichlorohydrin and bisulfite, such as, for example, Cocamidopropyl Hydroxysultaine, Coco-hydroxysultaine, Lauramidopropyl Hydroxysultaine or Lauryl Hydroxysultaine are obtained.

R⁴—N⁺(CH₃)₂—CH₂—CH(OH)—CH₂—SO₃⁻ (IIIa)

R⁵—C(O)—NH—CH₂—CH₂—CH₂—N⁺(CH₃)₂—CH₂—CH(OH)—CH₂—SO₃⁻ (IIIb)

where R⁴ is an alkyl or alkylamidoalkyl radical. The alkyl radical here may be branched or unbranched, preferably unbranched, and has preferably from 8 to 18, preferably from 10 to 16 and particularly preferably 10 to 14, carbon atoms. Particularly preferred hydroxysultaines are e.g. lauryl-hydroxysultaine, tallowamidopropylhydroxysultaine, erucamido-propylhydroxysultaine, alkyl ether hydroxypropylsultaine, cocoamidopropylhydroxysultaine, laurylamidopropylhydroxy-sultaine and cocoamidopropylhydroxysultaine and R⁵ is as defined above.

A further group of suitable amphoteric surfactants is the group of the compounds (and salts thereof), represented by formulae (IVa) and (IVb).

R⁵—C(O)—N(R⁶)—CH₂—CH₂—N(CH₂—CH₂—OH)—CH₂—COOH (IV)

R⁵—C(O)—N(CH₂—CH₂OH)—CH₂—CH₂—N(CH₂—COOH)₂ (IV)

where
R⁵=branched or unbranched, preferably unbranched alkyl radicals having 7 to 17 carbon atoms and
R⁶=H or CH₂—COOH, preferably H.

Preferred compounds of the formula IV are e.g. the reaction products of imidazoline derivatives with chloroacetic acid, in particular amphoacetates and amphodiacetates. Particularly preferred compounds of the formula IV are cocoamphoacetates or lauroamphoacetates, and also the corresponding diacetates. Preferred compounds of the formulae (IVa) and (IVb) are in particular sodium cocoamphoacetate, sodium lauroamphoacetate, disodium cocoamphodiacetate and disodium lauroamphodiacetate.

Suitable amphoteric surfactants are also C₈₋₁₈ fatty amphocarboxypropionates and C₈₋₁₈ fatty amphopropionates or fatty amine oxides, such as e.g. lauryldimethylamine oxide.

Particularly preferably, the compositions according to the invention comprise, as amphoteric surfactants, betaines, amphoacetates, amphodiacetates or amphopropionates selected from N-alkyl-N,N-dimethylammonium glycinates (alkylbetaines), for example cocosalkyldimethylammonium glycinate, N-acylamino-propyl-N,N-dimethylammonium glycinates (amidopropylbetaine), for example cocosacylaminopropyldimethylammonium glycinate (CAPB), 2-alkyl-3-carboxylmethyl-3-hydroxyethylimidazolines having in each case 8 to 18 carbon atoms in the alkyl or acyl group and cocosacylaminoethyl hydroxyethylcarboxymethyl-glycinate (cocoamphomonoacetate) (cocoamphomonoacetate) or mixtures thereof.

Besides the amphoteric surfactants, the composition according to the invention can optional comprise further surfactants. Such surfactants can be e.g. anionic surfactants, such as e.g. acyl isethionates, alkyl sulfates, alkyl ethoxysulfates, acyl sarcosinates, alkyl taurates and various amino acid-based amidocarboxylates, nonionic surfactants such as e.g. alcohol ethoxylates, where these must not be identical to the aforementioned OH-functional component, fatty amides, alkyl (poly)saccharides and alkylglucamides, and cationic surfactants, such as e.g. long-chain fatty amines, which may be optionally alkoxylated, in particular ethoxylated. Preferably, the composition according to the invention has one or more C₁₀-22-, preferably C₁₂₋₁₄-alkyl ethoxysulfate surfactants which preferably have on average 1 to 10, preferably 2 to 5, ethylene oxide units.

Preferred cationic surfactants are in particular quaternary ammonium compounds, in particular those provided with at least one linear and/or branched, saturated or unsaturated alkyl chain having 8 to 22 carbon atoms, such as e.g. alkyl-trimethylammonium halides, such as e.g. cetyltrimethylammonium chloride or bromide or behenyltrimethylammonium chloride or dialkyldimethylammonium halides, such as e.g. distearyldi-methylammonium chloride. Further preferred cationic surfactants are in particular monoalkylamidoquats, such as e.g. palmitamidopropyltrimethylammonium chloride, or corresponding dialkylamidoquats, readily biodegradable quaternary fatty acid esters based on mono-, di- or triethanolamine, methyldi-ethanolamine or alkylguanidinium salts.

Preferred nonionic surfactants are in particular addition products of from 2 to 100 mol of ethylene oxide and/or 0 to 5 mol of propylene oxide onto linear fatty alcohols having 8 to 22 carbon atoms (where these must not be identical to the aforementioned OH-functional component), onto fatty acids having 12 to 22 carbon atoms and onto alkylphenols having 8 to 15 carbon atoms in the alkyl group, C_{12/18}-fatty acid mono- and diesters of addition products of from 1 to 100 mol of ethylene oxide onto glycerol, glycerol mono- and diesters and sorbitan mono- and diesters of saturated and unsaturated fatty acids having 6 to 22 carbon atoms and ethylene oxide addition products thereof, alkyl mono- and oligoglycosides having 8 to 22 carbon atoms in the alkyl radical and ethylene oxide addition products thereof, addition products of from 2 to 200 mol of ethylene oxide onto castor oil and/or hydrogenated castor oil, partial esters based on linear, branched, unsaturated or saturated C₆-C₂₂-fatty acids, ricinoleic acid, and 12-hydroxystearic acid and glycerol, polyglycerol, pentaerythritol, dipentaerythritol, sugar alcohols (e.g. sorbitol), alkyl glucosides (e.g. methyl glucoside, butyl glucoside, lauryl glucoside), and polyglucosides (e.g. cellulose), mono-, di- and trialkyl phosphates, and also mono-, di- and/or tri-PEG alkyl phosphates and salts thereof, polysiloxane-polyether copolymers (dimethicone copolyols), such as e.g. PEG/PPG-20/6 dimethicone, PEG/PPG-20/20 dimethicone, bis-PEG/PPG-20/20 dimethicone, PEG-12 or PEG-14 dimethicone, PEG/PPG-14/4 or 4/12 or 20/20 or 18/18 or 17/18 or 15/15, polysiloxane-polyalkyl-polyether copolymers and corresponding derivatives, such as e.g. lauryl or cetyl dimethicone copolyols, in particular cetyl PEG/PPG-10/1 dimethicone (ABIL® EM 90 (Evonik Degussa)), mixed esters of pentaerythritol, fatty acids, citric acid and fatty alcohol according to DE 11 65 574 and/or mixed esters of fatty acids having 6 to 22 carbon atoms, methylglucose and polyols, such as e.g. glycerol or polyglycerol, citric acid ester such as e.g. glyceryl stearate citrate, glyceryl oleate citrate and dilauryl citrate.

The anionic surfactants are preferably selected from surfactants which contain water-solubilizing anionic groups, such as e.g. a carboxylate, sulfate, sulfonate or phosphate groups and a lipophilic radical. Skin-compatible anionic surfactants are known to the person skilled in the art in large numbers and are commercially available. These may be alkyl sulfates or alkyl phosphates in the form of their alkali metal, ammonium or alkanolammonium salts, alkyl ether sulfates, alkyl ether carboxylates, acyl sarcosinates, and acyl glutamates in the form of their alkali metal or ammonium salts.

Preferred optional surfactants are in particular alkyl ethoxysulfates of the formula (V)

R⁷—(O—CH₂—CH₂—)ₓ—OSO₃M (V)

where
R⁷=branched or unbranched, preferably unbranched, alkyl group which has preferably from 8 to 24, particularly preferably from 10 to 22 and very particularly preferably from 12 to 15, carbon atoms,
x=average number of ethylene oxide units per molecule, where x is preferably from 0.5 to 10, preferably from 1 to 5 and particularly preferably from 2 to 3, and
M=as defined above.

The mass ratio of optional surfactants, preferably alkyl ethoxysulfate surfactants to sulfosuccinate surfactants is preferably from 1:4 to 2:1, preferably 1:2.5 to 1:1.5.

Particularly preferred optional surfactants are in particular alkyl ethoxysulfates of the formula (V) are in particular C₁₀₋₂₂ alkyl ethoxysulfate surfactants which preferably have 1 to 5 ethylene oxide units.

The total content of surfactants in the composition according to the invention is preferably from 1 to 30, preferably 3 to 20 and particularly preferably from 6 to 15% by mass.

It may be advantageous if the composition according to the present invention has further OH-functional compounds which are not surfactants and which are different from those compounds (iii.)) used to produce the sulfosuccinate surfactants, Such OH-functional compounds can be e.g. monohydric alkanols, such as e.g. isopropanol or ethanol, glycerol. These further OH-functional compounds are preferably present in a fraction of at least 0.1% by mass and at most 10% by mass, based on the mass of the composition.

Moreover, the composition according to the invention can have one or more conditioners. These can be selected from the silicon-based or the non-silicon-based conditioners. Preferably, the composition according to the invention has one silicone, preferably selected from the group comprising organosilicones, amino-functional organosilicones, amino-functional organosilicone-polyether copolymers, quat-functional organosiloxanes, betaine-functional siloxanes and mixtures thereof, preferably in a concentration of from 0.01-5% by mass, particularly preferably 0.1-3% by mass.

The conditioners can be present as particles or as drops in the composition according to the invention. They may be liquid, semi-solid or solid, with the proviso that they are present in dispersed form in the composition, in a substantially complete and uniform manner. Preferably, the conditioners are in the form of liquid drops in the composition.

Silicone-based conditioners can be selected e.g. from the group comprising: polydiorganosiloxanes, in particular polydimethyl-siloxanes (dimethicone), polydimethylsiloxanes with hydroxyl end groups (dimethiconol), silicone resins, as described e.g. in WO 96/31188 and supplied by General Electric under the names GE SS4230 and GE SS4267, silicones (polysiloxanes) with a refractive index of at least 1.46 and at most 1.70, which have e.g. aryl-containing substituents. The fraction of silicone-based conditioners based on the composition according to the invention is preferably 0.01 to 5, preferably 0.1 to 3% by mass.

Particularly preferred non-silicone-based conditioners are e.g. organic quaternary compounds such as cetrimonium chloride, dicetyldimonium chloride, behentrimonium chloride, distearyl-dimonium chloride, behentrimonium methosulfate, distearoyl-ethyldimonium chloride, palmitamidopropyltrimonium chloride, guar hydroxypropyltrimonium chloride, hydroxypropylguar hydroxypropyltrimonium chloride, polyquaternium-10 or quaternium-80 or else amine derivatives such as e.g. stearamidopropyldimethylamine. The fraction of non-silicone-based conditioners based on the composition according to the invention is preferably 0.01 to 5, preferably 0.1 to 3% by mass.

As regards further suitable conditioners, reference is made to EP 1771538, which, in its entirety, forms part of the disclosure of the present invention.

It may be advantageous if the composition according to the invention has one or more cationic polymers. The cationic polymers have the advantage that they are likewise suitable as conditioners. The fraction of the cationic polymers is for example from 0.01 to 2, preferably 0.1 to 0.6 and particularly preferably from 0.15 to 0.45% by mass.

The cationic polymers preferably comprise cationic nitrogen-containing groups, such as e.g. quaternary ammonium or protonated amino groups. A nonlimiting list of examples of such polymers is described e.g. in CTFA Cosmetic Ingredient Dictionary, 6th edition, edited by Wenninger, J A and McEwen Jr, G N, (The Cosmetic, Toiletry, and Fragrance Association, 1995), the disclosure of which, in its entirety, forms part of the disclosure of the present application.

Preferred cationic polymers are selected from the group comprising cationically modified starch, cationically modified cellulose, cationically modified galactomannans, such as e.g. guar, cationically modified polyacrylates such as e.g. polyquaternium-7 and mixtures thereof.

Suitable cationic cellulose polymers are sold by Amerchol Corp. (Edison, N.J.), e.g. in its product series POLYMER JR and LR. Particularly suitable cationic cellulose polymers are e.g. polyquaternium 10 or polyquaternium 24, which is sold by Amerchol Corp. (Edison, N.J.) under the trade name polymer LM-200. A suitable and particularly preferred cationic guar derivative is in particular guar hydroxypropyltrimonium chloride, which is sold by Rhodia Corporation under the names JAGUAR EXCEL or JAGUAR C13S, Further suitable cationic polymers can be found e.g. in U.S. Pat. No. 3,958,581 and EP 1771538.

The composition according to the invention preferably has a viscosity of from 10 to 20 000 mPas, preferably from 1000 to 7000 and particularly preferably 2000 to 4500 mPas (determined with Brookfield LVF, spindle 3, 5 rpm, 25° C.). If necessary, the viscosity can be adjusted e.g. by adding one or more thickeners to the composition according to the invention.

It may be advantageous if the compositions according to the invention have one or more thickeners (viscosity regulators). Suitable thickeners are, for example, polysaccharides, in particular xanthan gum, guar, agar-agar, alginates and tyloses, carboxymethylcellulose, hydroxypropylmethylcellulose and hydroxyethylcellulose, also higher molecular weight poly-ethylene glycol mono- and diesters of fatty acids, poly-acrylates, (e.g. Carbopol™ or Synthalen™), polyacrylamides, polyvinyl alcohol and polyvinylpyrrolidone, surfactants such as, for example, ethoxylated fatty acid glycerides, esters of fatty acids with polyols such as, for example, pentaerythritol or trimethylolpropane, fatty alcohol ethoxylates with a narrowed homolog distribution or alkyl oligoglucosides, and also electrolytes such as sodium chloride and ammonium chloride. Thickeners which may be present are in particular also low molecular weight nonionic surfactants, such as cocoamide DEA/MEA and laureth-3, or polymeric, high molecular weight, associative, highly ethoxylated fatty derivatives, such as PEG-200 hydrogenated glyceryl palmate, PEG-120 methyl glucose dioleate (Antil® 120 Plus), PEG-55 propylene glycol oleate, PEG-18 glyceryl oleate/cocoate.

Furthermore, thickeners which can be used are e.g. waxes, such as hydrogenated castor wax, beeswax or microwax, inorganic thickeners, such as e.g. silica, alumina or sheet silicates (e.g. hectorite, laponite, saponite), which may be hydrophobically modified, aerosils, sheet silicates and/or metal salts of fatty acids, such as e.g. zinc stearate.

As thickeners, particular preference is given to using compounds from the class of the hydrophobically modified, water-soluble nonionic polyols. Preferred compounds of this class are e.g. PEG-120 methyl glucose dioleate, which is sold e.g. under the trade name Antil® 120 Plus by Evonik Goldschmidt GmbH, PEG-150 pentaerythrityl tetrastearate, which is sold e.g. by Croda under the trade name CROTHIX®, PEG-75 dioleate, which is sold e.g. by Kessco under the trade name PEG-4000 DIOLEATE, or PEG-150 distearate, which is sold e.g. by Evonik Goldschmidt under the trade name Rewopal® PEG 6000 DS.

As a result of the fraction of OH-functional compound in the composition according to the invention, the fraction of required thickener can be reduced. Preferred compositions which have a viscosity in the aforementioned range preferably have from 0.1 to 10, preferably from 0.5 to 8 and particularly preferably from 1 to 7, % by mass of thickeners, in particular of the aforementioned thickeners and particularly preferably of the hydrophobically modified, water-soluble nonionic polyols.

Particularly preferred compositions according to the invention are those in which two or more of the aforementioned particularly advantageous additives are present at the same time.

The compositions according to the invention can be used for producing, or as, cleansing or care compositions, in particular for the cleansing or care of skin and hair.

Accordingly, the present invention further provides skin and/or hair care or cleansing compositions which have a composition according to the invention.

Depending on the intended use, the compositions according to the invention can comprise customary further constituents in customary concentrations. The further constituents can be selected e.g. from emollients, emulsifiers, thickeners/viscosity regulators/stabilizers, UV photoprotective filters, antioxidants, hydrotropes (or polyols), solids and fillers, film formers, pearlescent additives, deodorant and antiperspirant active ingredients, insect repellents, self-tanning agents, preservatives, conditioners, perfumes, dyes, biogenic active ingredients, care additives, super fatting agents and solvents. A list of substances which may be customary for these constituents and preferred within the context of the present invention can be found e.g. in DE 102005011785 and EP 2000124.

The examples below describe, by way of example, the subject matter of the present invention, without any intention to limit the invention to these embodiments.

### EXAMPLES

### Preparation Example

### Preparation of a Sulfosuccinate Solution

The preparation of the sulfosuccinate solutions used is carried out in accordance with comparative example 1 of DE 44 14 863. Instead of Dehydol®, the OH-functional component used was an equivalent amount of ethoxylated fatty alcohol of the formula VI

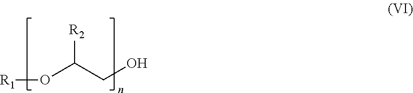

where R²=H, R¹ to 0.8% by mass C₁₀-alkyl, to 74% by mass C₁₂-alkyl, to 24% by mass C₁₄-alkyl and to 1.2% by mass C₁₆-alkyl, and to 21% by mass n=0, to 16% by mass n=1, to 15% by mass n=2, to 14% by mass n=3, to 11% by mass n=4, to 8% by mass n=5, to 6% by mass n=6, to 4% by mass n=7 and remaining % by mass (to 100%) n>=8 (determined by GC). After the work-up carried out as described in comparative example 1 of DE 44 14 863, a sulfosuccinate solution was obtained which had a content of sulfosuccinate of ca. 33% by mass. This solution was used in the examples described below.

### Example 1

### Preparation of Compositions According to the Invention

Experiments for the thickening of cleansing compositions were carried out. The compositions of the formulations are listed in table 1 (unless stated otherwise, data are in % by mass). Texapon NSO-IS, TEGO® Betain F 50 and water were mixed with stirring and admixed at 25° C. with the sulfosuccinate solution (33% strength by mass aqueous solution), which had the content of fatty alcohol ethoxylate stated in table 1. The Antil® 120 Plus was added in the form of pellets and the mixture was heated to 50° C. with stirring for 5 min (until the pellets completely dissolved). The solution was then heat-treated overnight at 25° C. and the viscosity was determined at 25° C. using a Brookfield LVF viscometer (spindle 3, 5 rpm). The results of the viscosity measurement are likewise given in table 1. The viscosities of the compositions as a function of the content of free OH-functional compound are shown in FIG. 1.

TABLE 1
Feed materials and results relating to example 1 (examples 1a
to g according to the invention, example 1h comparative example)

| Feed materials | Ex. 1a | Ex. 1b | Ex. 1c | Ex. 1d | Ex. 1e | Ex. 1f | Ex. 1g | Ex. 1h |
|---|---|---|---|---|---|---|---|---|
| Sulfosuccinate | 9.10% | 9.10% | 9.10% | 9.10% | 9.10% | 9.10% | 9.10% | 9.10% |
| 33% | | | | | | | | |
| Texapon NSO-IS | 21.40% | 21.40% | 21.40% | 21.40% | 21.40% | 21.40% | 21.40% | 21.40% |
| 28% | | | | | | | | |
| Tego Betain F | 7.90% | 7.90% | 7.90% | 7.90% | 7.90% | 7.90% | 7.90% | 7.90% |
| 50 38% | | | | | | | | |
| Antil ® 120 | 2.50% | 2.50% | 2.50% | 2.50% | 2.50% | 2.50% | 2.50% | 2.50% |
| Plus | | | | | | | | |
| Water | 59.10% | 59.10% | 59.10% | 59.10% | 59.10% | 59.10% | 59.10% | 59.10% |
| FAEO in % | 2.00 | 1.50 | 2.40 | 2.80 | 1.90 | 3.20 | 3.50 | 10.20 |
| (based on | | | | | | | | |
| sulfosuccinate | | | | | | | | |
| solution) | | | | | | | | |
| FAEO in % | 0.18 | 0.14 | 0.22 | 0.25 | 0.17 | 0.29 | 0.32 | 0.93 |
| (based on | | | | | | | | |
| formulation) | | | | | | | | |
| FAEO in % | 6.1 | 4.5 | 7.3 | 8.5 | 5.8 | 9.7 | 10.6 | 30.9 |
| based on | | | | | | | | |
| sulfosuccinate | | | | | | | | |
| Appearance | clear | clear | clear | clear | clear | clear | clear | clear |
| Viscosity in | 1400 | 1390 | 1500 | 1470 | 1090 | 1900 | 2260 | 45000 |
| mPas | | | | | | | | |

FAEO: fatty alcohol ethoxylate
Texapon ® NSO-IS is a 28% strength by weight aqueous solution of sodium lauryl ether sulfate, sold by Cognis
Tego ® Betain F50 is a 38% strength by weight solution of cocamidopropylbetaine in water, sold by Evonik Goldschmidt GmbH.

Antil® 120 Plus is PEG-120 methylglucose dioleate, sold by Evonik Goldschmidt GmbH.

### Example 2

### Comparison of the Required Amount of Thickener

Experiments were carried out to determine the required amount of thickener for establishing a formulation viscosity of 3500 mPas. The preparation of the formulations and also the viscosity measurements were carried out as stated in example 1. For examples 2b and 2c, the sulfosuccinate solution was admixed, prior to the preparation of the formulation, with the stated amount of free OH-functional compound and thoroughly mixed using a magnetic stirrer at 40° C. until a clear solution was formed. Instead of Antil® 120 Plus, Antil® 171 (PEG-18 glyceryl oleate/cocoate, Evonik Goldschmidt GmbH) was used. The fraction of Antil® 171 was adjusted such that the formulation had a viscosity of 3500 mPas at 25° C. The composition of the formulations used and also the results can be found in table 2. It is clear to see that as a result of the compositions according to the invention, the required amount of thickener was able to be reduced.

TABLE 2
Formulations and results of example 2.

| | Example | | |
|---|---|---|---|
| | 2a | 2b | 2c |
| Sulfosuccinate 33% | 9.1% | 9.1% | 9.1% |
| Texapon NSO-IS 28% | 21.4% | 21.4% | 21.4% |
| TEGO ® Betain F 50 38% | 7.9% | 7.9% | 7.9% |
| Fatty alcohol EO in % by | 0.0 | 1.0 | 2.0 |
| weight (based on | | | |
| sulfosuccinate solution) | | | |
| Antil ® 171 | 6.50% | 6.23% | 6.13% |
| Water | ad 100% | ad 100% | ad 100% |
| % reduction in the | 0.0 | 4.2 | 5.7 |
| thickener fraction based | | | |
| on 2a | | | |

### Example 3

### Comparison of the Viscosities

Formulations were prepared analogously to example 2 with different contents of fatty alcohol ethoxylate, although the fraction of thickener (Antil® 171) was kept constant at 6.0%. The viscosity at 25° C. was measured as in ex. 1 and 2. The composition of the formulations used and also the results can be found in table 3. It is clear to see that the formulation viscosity of the compositions according to the invention is increased as result of adding free, OH-functional compound.

TABLE 3
Formulations and results of example 3.

| | Example | | |
|---|---|---|---|
| | 3a | 3b | 3c |
| Sulfosuccinate 33% | 9.1% | 9.1% | 9.1% |
| Texapon NSO-IS 28% | 21.4% | 21.4% | 21.4% |
| TEGO ® Betain F 50 38% | 7.9% | 7.9% | 7.9% |
| Fatty alcohol EO in % (based | 0.0 | 1.0 | 2.0 |
| on sulfosuccinate solution) | | | |
| Antil ® 171 | 6.0% | 6.0% | 6.0% |
| Water | ad 100% | ad 100% | ad 100% |
| Viscosity/mPas | 1800 | 2400 | 3000 |

### Example 4

### Experiments Relating to the Foaming Behavior

Experiments relating to the foaming behavior of cleansing compositions were carried out. Formulations 4a, 4b and 4c (0.5% in water, 10° German hardness, 30° C., pH 6) were prepared analogously to examples 2 and 3 (but without the addition of a thickener) and analyzed using a SITA-Foam Tester R-2000 (Software: SITA-Foam DAC/DL; volumes/measurement 300 ml; 1500 rpm; five-fold measurement). The foam volume after 20 s was measured. The composition of the formulations used and also the results can be found in table 4. It is clear to see that the addition of free, OH-functional compound leads to an increase in foam volume and thus to a better foaming behavior.

TABLE 4
Formulations and results of example 4.

| | Example | | |
|---|---|---|---|
| | 4a | 4b | 4c |
| Sulfosuccinate 33% | 9.1% | 9.1% | 9.1% |
| Texapon NSO-IS 28% | 21.4% | 21.4% | 21.4% |
| TEGO ® Betain F 50 38% | 7.9% | 7.9% | 7.9% |
| Fatty alcohol EO in % | 0.0 | 1.0 | 2.0 |
| (based on sulfosuccinate | | | |
| solution) | | | |
| Water | ad 100% | ad 100% | ad 100% |
| Foam volume/ml after | 467 | 481 | 491 |
| 20 s | | | |

1. An aqueous composition comprising:
i) from 0.5 to 10% by mass of at least one sulfosuccinate surfactant,
ii) from 0.5 to 10% by mass of at least one amphoteric surfactant, and
iii) free, OH-functional compounds, where these are like the alcohol component of the esters of the sulfosuccinate surfactants used,
where the mass ratio of sulfosuccinate surfactant i) to free, OH-functional compound is from 100:1 to 100:20.

2. The composition as claimed in claim 1, wherein the OH-functional compound is selected from the group comprising alcohols having 10 to 14 carbon atoms or polyether alcohols of these alcohols.

3. The composition as claimed in claim 1, wherein the OH-functional compound has polyether groups having on average 1 to 10 alkoxy units.

4. The composition as claimed in claim 1, wherein the OH-functional compound is selected from the group comprising lauryl alcohol, myristyl alcohol, PEG-3 lauryl alcohol, PEG-3 myristyl alcohol or a mixture of PEG-3 alkyl alcohols, where the mixture has less than 2 mol % of alkyl alcohols having 10 or fewer carbon atoms in the alkyl chain, from 65 to 75 mol % of alkyl alcohols having 12 carbon atoms in the alkyl chain, from 20 to 30 mol % of alkyl alcohols having 14 carbon atoms in the alkyl chain and less than 2 mol % of alkyl alcohols having 16 or more carbon atoms in the alkyl chain.

5. The composition as claimed in claim 1, wherein the amphoteric surfactant is selected from the group comprising betaines, amphoacetates, amphodiacetates, amphopropionates, hydroxysultaines, amine oxides and mixtures thereof.

6. The composition according to claim 5, wherein the betaine is a C₈₋₁₈ alkylbetaine or a C₈₋₁₈ alkylamidopropylbetaine or a mixture thereof.

7. The composition as claimed in claim 1, wherein said composition comprises a C₈₋₂₂ alkyl ethoxysulfate surfactant.

8. The composition as claimed in claim 7, wherein the mass ratio of alkyl ethoxysulfate surfactant to sulfosuccinate surfactant is from 1:4 to 2:1.

9. The composition as claimed in claim 1, wherein the composition has further OH-functional compounds which are different from those OH-functional compounds iii) used for producing the sulfo-succinate surfactants.

10. The composition as claimed in claim 9, wherein the further OH-functional compounds are present in a fraction of at least 0.5% by mass, based on the mass of the composition.

11. The composition as claimed in claim 1, wherein said composition comprises a silicon selected from the group comprising organosilicones, amino-functional organosilicones, amino-functional organo-silicone-polyether copolymers and mixtures thereof.

12. The composition as claimed in claim 1, wherein said composition comprises a cationic polymer selected from cationically modified polysaccharides of the group comprising cationically modified starch, cationically modified cellulose, cationically modified galactomannans, cationically modified polyacrylates and mixtures thereof.

13. The composition as claimed in claim 1, wherein said composition has a pH of from 4 to 7.

14. The composition as claimed in claim 1, wherein said composition has a viscosity of from 1000 to 7000 mPas.

15. (canceled)

16. A skin and/or hair cleansing composition, comprising the composition as claimed in 1.
